(19) United States
(12) Patent Application Publication
(10) Pub. No.: US 2018/0015054 A1
(43) Pub. Date: Jan. 18, 2018

(54) Method of Maintaining the Anti-Depressant Effect of Ketamine With Lithium

(71) Applicant: Icahn School of Medicine at Mount Sinai, New York, NY (US)

(72) Inventors: Dennis S. Charney, Chappaqua, NY (US); James Murrough, New York, NY (US)

(21) Appl. No.: 15/541,099

(22) PCT Filed: Dec. 28, 2015
(86) PCT No.: PCT/US2015/067673

§ 371 Date: Jun. 30, 2017

Related U.S. Application Data
(60) Provisional application No. 62/098,403, filed on Dec. 31, 2014.

(51) Int. Cl. A61K 31/135; A61K 33/00

(52) U.S. Cl. CPC A61K 31/135; A61K 33/00

(57) ABSTRACT

Disclosed herein is method for maintaining the anti-depressant effect of ketamine by administering lithium to patients that respond to ketamine treatment.

### FIELD OF THE INVENTION

The present invention relates to methods of treating patients afflicted with major depressive disorder, treatment resistant depression, bipolar depression and post-traumatic stress disorder.

### BACKGROUND OF THE INVENTION

All current antidepressants target aspects of the monoamine neurotransmitter systems and a major goal of current neuropharmacology research is to identify safe and more effective treatments for depression by targeting neural systems and chemical messengers outside of the monoamine system. Converging evidence from in vivo brain imaging studies, post-mortem investigations, and gene expression studies implicate abnormalities in amino acid neurotransmitter systems in the pathophysiology of major depressive disorder (MDD) i.e. severe symptoms that interfere with the patient's ability to work, sleep, study, eat, and enjoy life. In this context, the discovery that the glutamate N-methyl-D-aspartate (NMDA) receptor antagonist ketamine is a rapidly acting antidepressant—even among patients with treatment resistant depression (TRD)—has enormous scientific and public health implications and has generated considerable enthusiasm among clinicians and patients alike.

Post-traumatic stress disorder (PTSD) is a prevalent and highly debilitating psychiatric disorder that is notoriously difficult to treat.

The treatment of PTSD is extremely challenging, and may include many years of individual and group therapy and medications such as antidepressants, anxiolytic drugs, alpha adrenergic antagonists, opiates, or cortisol with variable results. Selective serotonin reuptake inhibitors (SSRIs) are currently recommended as the first-line pharmacotherapy. However, up to 40% of SSRI-treated PTSD patients do not respond and >70% never achieve full remission. The two SSRIs that are approved for PTSD by the United States Food and Drug Administration (FDA), paroxetine and sertraline, have modest effect sizes and limited efficacy in all three clusters of illness: re-experiencing, avoidance and numbing, and hyperarousal. The severity and significance of lack of SSRI efficacy, especially in light of the observed relationship between trauma exposure and increased rates of disability, unemployment, and social assistance highlights the urgent need for novel pharmacological interventions targeting the core pathophysiology of PTSD.

Bipolar disorder, also known as manic-depressive illness, is a brain disorder that causes unusual shifts in mood, energy, activity levels, and the ability to carry out daily tasks. Symptoms of bipolar disorder can be severe. The extreme “mood swings” between mania and depression include emotional highs (mania or hypomania) and lows (depression) and can occur rapidly. The depression component of bipolar disorder is treated with antidepressant medications including for example selective serotonin reuptake inhibitors (SSRI's). As used herein, the term bipolar depression (BPD) refers to the depressive component of bipolar disorder.

Ketamine is an N-methyl-D-aspartate (NMDA) glutamate receptor antagonist that is currently approved as an anesthetic agent in the U.S. More recently, a growing body of literature supports the rapid antidepressant effect of ketamine in patients with BPD, TRD, MDD and PTSD. The onset of antidepressant effects of ketamine has been reported as early at 40 minutes following a single intravenous administration and peak antidepressant effects have been reported between 4 and 72 hours following treatment.

The intranasal administration of ketamine to patients afflicted with TRD is disclosed in U.S. Pat. No. 8,785,500 (the disclosure of which is incorporated by reference herein in its entirety).

A patient is categorized as suffering from TRD if the patient suffers from depression but has not responded to at least two adequate antidepressant treatments including by way of non-limiting example serotonin reuptake inhibitors e.g., fluoxetine, venlafaxine, and duloxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, a-methyltyrosine, reserpine, tetrabenazine, benztropine, pargyline; noradrenergic agonists and antagonists e.g., clonidine, phenoxybenzamine, phentolamine, tropolone, as well as non-pharmaceutical treatments such as electroconvulsive therapy (ECT).

MDD is characterized by dysfunctional processing of social, emotional, and reward-related information, leading to the cardinal clinical symptoms of pervasive depressed mood, anhedonia (e.g., reduced capacity to experience pleasure), and a negative cognitive bias. In particular, neuropsychological and neuroimaging investigations have confirmed a negative emotion processing bias as a central feature of MDD. Patients with MDD demonstrate increased attention and memory for negative social information (e.g., pictures of human facial expressions) and a bias away from positive information. For example, depressed patients show a bias away from positive facial expressions and require a greater intensity of emotional expression to correctly identify happy (but not sad) emotion.

PTSD is characterized by flashbacks, emotional numbness, and insomnia, and is associated with functional impairments, physical health concerns, and mental health comorbidities, such as depression, with six fold higher risk of suicide. PTSD can result from a catastrophic and threatening event, e.g., a natural disaster, wartime situation, accident, domestic abuse, or violent crime. Symptoms typically develop within three months, but can emerge years after the initial trauma. At some point in their lifetimes, 5-8% of men and 10-14% of women, generally suffer from PTSD.

A bipolar disorder diagnosis is made by taking careful note of the patient's symptoms, including their severity, length, and frequency. Day to day “mood swings” do not necessarily lead to a diagnosis of bipolar disorder. Instead, the diagnosis depends on observing periods of unusual elevation or irritability in mood, coupled with increases in energy, sleeplessness, and fast thinking or speech. The patient's symptoms are assessed using the criteria set forth in the American Psychiatric Association text entitled Diagnostic and Statistical Manual of Mental Disorders or DSM-IV.

In an initial study of 26 patients with treatment-resistant depression (TRD) treated with a single open-label intravenous (IV) infusion of ketamine (0.5 mg/kg), 65% showed significant alleviation of the depression [defined as a reduction of 50% or more on the Montgomery-Asperg Depression Rating Scale, (MADRS)] Mathew S J, Murrough J W, aan het Rot M, Collins K A, Reich D L, Charney D S. Riluzole for relapse prevention following intravenous ketamine in treatment-resistant depression: a pilot randomized, placebo-controlled continuation trial. The international journal of neuropsychopharmacology I official scientific journal of the Collegium Internationale Neuropsychopharmacologicum. February 2010; 13(1):71-82.

Recent studies of ketamine in depression examined the efficacy of ketamine in TRD. In one study, Antidepressant efficacy of ketamine in treatment-resistant major depression: a two-site randomized controlled trial. Murrough J W, Iosifescu D V, Chang L C, Al Jurdi R K, Green C E, Perez A M, Iqbal S, Pillemer S, Foulkes A, Shah A, Charney D S, Mathew S J. Am J Psychiatry. 2013 October; 170 (10):1134-42, 72 patients received a single intravenous (IV) infusion of either ketamine hydrochloride (0.5 mg/kg) or midazolam (0.045 mg/kg) in a 2:1 randomization under triple-masked conditions (patient, rater, and anesthesiologist blind to treatment). The primary outcome was change in depression severity as measured by the Montgomery-Asberg Depression Rating Scale (MADRS) from baseline to 24 hours post-infusion. Patients in the ketamine group had significantly greater improvement in the MADRS score at 24 hours compared to the midazolam group [t (68)=3.34, P<0.001. Notably, ketamine no longer separated from placebo at the 7-day time point, suggesting a 1-week duration of efficacy following a ketamine infusion. Ketamine was very safe and well tolerated. No patient experienced dissociative or psychotomimetic effects beyond 2 hours. The aforementioned study and A randomized trial of an N-methyl-D-aspartate antagonist in treatment-resistant major depression. Zarate C A Jr, Singh J B, Carlson P J, Brutsche N E, Ameli R, Luckenbaugh D A, Charney D S, Manji H K. Arch Gen Psychiatry. 2006 August; 63(8):856-64 clearly demonstrate a one week duration of action of ketamine, i.e., anti-depressant effect endures for about one week after administration. The discovery of ketamine as a mechanistically novel, rapid acting antidepressant in humans has spurred a series of preclinical studies investigating the antidepressant mechanism of ketamine. Low-dose ketamine increases glutamate signaling in prefrontal cortical regions and initiates a cascade of molecular events resulting in synaptogenesis and enhanced synaptic functioning. Signaling pathways linked to the action of ketamine include the BDNF-tropomyosin related kinase B (TrkB) pathway and GSK3 associated pathways, among others. Overall, ketamine appears to rapidly reverse neuronal atrophy and synaptic deficits in cortical and hippocampal neurons caused by chronic stress and induction of synaptogenesis is the putative molecular mechanism underlying ketamine's antidepressant effects.

The rapid and robust antidepressant effects of ketamine, unfortunately, are relatively short-lived, and little research to date has investigated strategies for maintaining the anti-depressant response to ketamine. The duration of antidepressant efficacy following a single administration of ketamine is anywhere from a few days to approximately a week or two.

The glutamate-release inhibitor riluzole was initially tested as a relapse prevention strategy following ketamine in TRD but the results established that riluzole was not superior to placebo in preventing relapse. In that study, the initial response rate was 65% and the cumulative risk of relapse over the 4-week observation period was 62%.

A second study conducted at the NIMH Intramural Program similarly found no benefit of riluzole beyond placebo for relapse prevention following ketamine and a cumulative probability of relapse during the 4-week assessment period of 73%. Ibrahim L, Diazgranados N, Franco-Chaves J, et al. Course of improvement in depressive symptoms to a single intravenous infusion of ketamine vs add-on riluzole: results from a 4-week, double-blind, placebo-controlled study. Neuropsychopharmacology 2012; 37: 1526-33.

Repeated ketamine infusions were subsequently tested over a 2-week period and risk of relapse measured following cessation of ketamine treatment in the absence of concomitant medication. As expected, a rapid improvement in depressive symptoms was observed within 24 hours of the first ketamine infusion. This improvement was then maintained throughout the remainder of infusions, with an overall response rate of 70.8%. Following the sixth and final ketamine infusion, the median time to relapse was 18 days. The cumulative risk of relapse over the 3-month observation period while free of concomitant medication was 70%. Murrough J W, Perez A M, Pillemer S, et al. Rapid and Longer-Term Antidepressant Effects of Repeated Ketamine Infusions in Treatment-Resistant Major Depression. Biol Psychiatry 2012; [ePub ahead of print]. Thus, research has established that ketamine is safe and effective in TRD over the short term (days-weeks) and that there is a high risk of relapse upon cessation of ketamine treatment.

Feder et al (JAMA Psychiatry 2014:71(6) 681-688 found that ketamine infusion was associated with significant and rapid reduction in PTSD symptom severity, compared with midazolam when assessed 24 hours after infusion. Greater reduction of PTSD symptoms following treatment with ketamine was evident in both crossover and first period analyses and remained significant after adjusting for baseline and 24 hour symptom severity.

Therefore, identifying safe and effective antidepressant continuation strategies following ketamine administration represents a critical area of need.

### SUMMARY OF THE INVENTION

Methods and compositions for the treatment of BPD, MDD, TRD and PTSD are described. More specifically, the invention demonstrates that administration of lithium to patients that have responded to treatment with ketamine is effective to maintain the anti-depressant efficacy of the ketamine treatment and to provide continued alleviation of the symptoms of BPD, MDD, TRD and PTSD respectively. This is beneficial because patients can self-administer lithium while administration of ketamine must be done by trained medical professionals.

In particular embodiments, the invention provides a method of treating a human patient for BPD, TRD, MDD or PTSD, by administering a composition comprising ketamine to the patient at a dosage effective to reduce or eliminate the symptoms of the BPD, TRD, MDD or PTSD, determining that the patient has responded to the ketamine treatment and thereafter administering a daily dose of lithium to the patient to maintain the ketamine response. Using this combined treatment, the ketamine anti-depressant effect may be maintained for up to several months or more after administration of ketamine has been discontinued.

Esketamine, an enantiomer of ketamine, S (+)-ketamine, a compound two times more potent than racemic (which is a mixture of the S (+) and R (+)) is also useful in practicing the methods of the present invention.

In more specific embodiments, the ketamine is in a pharmaceutically acceptable carrier and is administered at a dose of between about 0.1 mg/kg per day to about 1.0 mg/kg/day, and following a determination that the patient has responded to the ketamine treatment (as determined for example by an at least fifty percent improvement in depression severity within twenty four hours after administration of ketamine using the Montgomery-Asberg Depression Rating Scale(MADRS)), administering lithium to the patient at a dose of between about 300 to about 1800 mg/day. The determination that a patient has responded to the ketamine treatment is made at least twenty-four hours after administration of the initial ketamine dose. The ketamine can be administered intra-nasally, intravenously, or via another acceptable route, such as the transdermal route.

### DETAILED DESCRIPTION OF THE INVENTION

Disclosed herein are various methods and compositions for treating TRD, MDD, PTSD and BPD using intranasal administration of ketamine and in an alternative embodiment, intravenous administration of ketamine.

The treatment of TRD, MDD, PTSD or BPD may be achieved through a method that comprises intranasal or intravenous administration of a single dose of the ketamine. Alternatively, multiple doses of ketamine may be administered. In specific embodiments, a single intranasal administration of an aerosol formulation of ketamine is sufficient to reduce the severity of the depression by fifty percent or more for 7 days or more.

At least twenty-four hours after administration of a first dose of ketamine the patient is tested to determine if the patient is responding to the ketamine. The response can be measured using any of the available tests for scoring a patients depression including for example the Montgomery-Asberg Depression Rating Scale (MADRS). Prior to beginning ketamine therapy the patient is tested using one of the standard depression rating scales .e.g. the MADRS. In the case of a patient with PTSD, the Clinician Administered PTSD Scale (CAPS) or a similar scale would be used to measure symptom severity. A patient is considered to be responding to ketamine if there is at least a fifty percent (50%) improvement in depression severity compared to the patients initial (pre-ketamine administration) test score. If the patient does not respond to the initial ketamine dose, administration of ketamine is continued one, two or three times per week until the patient shows at least a fifty percent improvement in depression severity. That is to say, the patient is observed after administration of each ketamine dose and a determination made as to whether the patient has responded to the ketamine.

Once it has been established that the patient is responding to ketamine, lithium (preferably in the form of lithium carbonate) is administered to the patient on a daily basis. Administration of lithium preferably begins on the day after the patient has received the last dose of ketamine or immediately after it has been established that the patient is responding to ketamine. The daily dose of lithium effective to maintain the ketamine antidepressant effect is between about 300 and 1800 mg/day and preferably between about 600 and 1200 mg/day and can be administered in a single daily dose or in divided doses. The lithium can be in tablet or capsule form or administered in a solution. Lithium is also available in an extended release form, which is suitable for this use in the invention. (US trade names for lithium carbonate and lithium carbonate-extended release include Eskalith, Eskalith-CR and Lithobid). In one embodiment, lithium and ketamine can be co-administered to the patient.

The therapeutic anti-depressant effect of ketamine may be maintained for several months or longer, as long as the lithium medication is continued. At some point the lithium may lose its maintenance effect. At this time the patient is again treated by administration of ketamine (via the intranasal or intravenous route) until it is determined that she is responding to treatment (.e.g. by measuring an improvement in the patient's depression of at least fifty percent using e.g. the MADRS) and then started again on lithium treatment.

The invention provides for administration of a therapeutically effective dose of ketamine, i.e., a dose effective to alleviate BPD, TRD, MDD or PTSD. The actual effective dose will vary, depending on the body weight of the patient, the severity of the depression, the route of administration, the nature of medications administered concurrently, the number of doses to be administered per day, and other factors generally considered by the ordinary skilled physician in the administration of drugs. In a specific embodiment, the effective amount of ketamine administered to a patient suffering from treatment-resistant depression is about 10% to about 20% of the amount used to induce anesthesia. In another specific embodiment, the effective dose of ketamine is about 0.01 mg per kg of body weight (0.01 mg/kg) to about 1 mg/kg; preferably about 0.05 mg/kg to about 0.7 mg/kg. In another specific embodiment intravenous ketamine is administered two or three times a week (on days 1, 3 and 5) at a dose of 0.5 mg/kg of body weight. The same dosage ranges may be used in treating patients with BPD, MDD or PTSD.

Preferably, the effective dose is titrated under the supervision of a physician or medical care provider, so that the optimum dose for the patient's specific condition and disease state accurately determined.

In one embodiment, the ketamine is introduced into the subject in the aerosol form in an amount between about 0.01 mg per kg body weight of the patient up to about 1.0 mg per kg body weight of the patient. In a specific embodiment ketamine is intra nasally administered three times per week (on days 1, 3 and 5) at a dose of 0.5 mg/kg of body weight. In another specific embodiment, the dosage is administered as needed. One of ordinary skill in the art can readily determine a volume or weight of aerosol corresponding to this dosage based on the concentration of ketamine in an aerosol formulation of the invention.

While it is possible to use a composition disclosed herein (e.g., a composition comprising ketamine for therapy) as is, it may be preferable to formulate the composition in a pharmaceutical formulation, e.g., in admixture with a suitable pharmaceutical excipient, diluent, or carrier selected with regard to the intended route of administration and standard pharmaceutical practice. Suitable carriers and their formulations are described in Remington: The Science and Practice of Pharmacy, 21st ed., 2005, Lippincott, Williams & Wilkins, Phila., Pa. Accordingly, in one aspect, a pharmaceutical composition or formulation comprises at least one active composition of ketamine in association with a pharmaceutically acceptable excipient, diluent, and/or carrier. The excipient, diluent and/or carrier must be “acceptable” in the sense of being compatible with the other ingredients of the formulation and not deleterious to the recipient thereof.

For in vivo administration to humans, the compositions can be formulated according to known methods used to prepare pharmaceutically useful compositions. Compositions may be designed to be short-acting, fast-releasing, long-acting, or sustained-releasing. Thus, pharmaceutical formulations may also be formulated for controlled release or for slow release.

When formulated in a pharmaceutical composition or formulation, ketamine can be admixed with a pharmaceutically acceptable carrier or excipient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Other exemplary carriers include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like.

In general, preparations according to this invention include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also optionally contain adjuvants, preserving, wetting, emulsifying, and dispersing agents. The pharmaceutical compositions may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

One preferred route of administration of ketamine is intravenous (IV). Ketamine may thus also be prepared in a formulation or pharmaceutical composition appropriate for IV administration. Ketamine can be admixed with a pharmaceutically acceptable carrier or excipient as described above. By way of example, ketamine can be formulated in a saline solution for intravenous administration.

A preferred mode of administration for ketamine is intranasal administration, i.e., through the nasal mucosa and through the nose-brain pathway directly into the cerebrospinal fluid. Ming Ming Wen, Discov Med, “Olfactory Targeting Through Intranasal Delivery of Biopharmaceutical Drugs to the Brain—Current Development,” 2011, 11:497-503, is hereby incorporated by reference in its entirety. As discussed in Wen, drugs administered intranasally may reach the brain via alternatives pathways. In one pathway, drugs, e.g., ketamine, are absorbed systemically, following absorption through the blood vessels of the nasal respiratory epithelium. Drugs delivered via this systemic pathway must first cross the blood brain barrier, prior to reaching the brain. In an alternative delivery pathway, drugs administered intranasally can be rapidly transported into the CNS via the connection between the olfactory epithelium at the roof of the nasal cavity and the trigeminal system of the brain. This affords a direct connection, with no synapse between the olfactory neurons and the brain. The pathway thus allows transport of active agents to the brain without passage through the blood brain barrier.

Excipients that may improve intranasal administration of ketamine include mucoadhesives (e.g., carbopol, carboxymethylcellulose, and hyaluronan), penetration enhancers that improve permeability and bioavailability of ketamine upon contact of the nasal mucosa (e.g., peppermint oil, N-tridecyl-beta-D-maltoside, and hexarelin). Chitosan, for example, has both mucoadhesive and penetration enhancing properties. Other agents that can be used to in formulations for intranasal delivery include liposomes (e.g., cationic liposomes and liposomes coated with polyethylene glycol (PEG), vasoconstrictors (e.g., phenylephrine), to limit absorption through the systemic pathway and increase absorption through the olfactory epithelium. Additional formulations and methods for intranasal administration are found in Ilium L., J Pharm Pharmacol, 56:3-17, 2004 and Ilium, L., Eur J Pharm Sci 11:1-18, 2000, each of which is hereby incorporated by reference in its entirety.

Either of liquid and powder intranasal formulations may be used. Ketamine, for example, may be combined with a dispersing agent, or dispersant, and administered intranasally in an aerosol formulation optimized for intranasal administration.

Intranasal liquid aerosol formulations contain ketamine and a dispersing agent in a physiologically acceptable diluent. Aerosolized formulations are broken down into liquid or solid particles in order to ensure that the aerosolized dose actually reaches the mucous membranes of the nasal passages. The term “aerosol particle” is used to describe the liquid or solid particle suitable for intranasal administration, i.e., that will reach the mucous membranes. Other considerations, such as construction of the delivery device, additional components in the formulation, and particle characteristics are important. These aspects of intranasal administration of a drug are well known in the art, and manipulation of formulations, aerosolization means and construction of a delivery device require at most routine experimentation by one of ordinary skill in the art.

Intranasal aerosol formulations can also be prepared as a dry powder formulation comprising a finely divided powder form of ketamine and a dispersant. For example, the dry powder formulation can comprise a finely divided dry powder containing ketamine, a dispersing agent and also a bulking agent. Bulking agents useful in conjunction with the present formulation include such agents as lactose, sorbitol, sucrose, or mannitol, in amounts that facilitate the dispersal of the powder from the device.

Nasal formulations may be administered with the aid of a delivery device, e.g., an aerosol delivery. Any form of aerosolization known in the art, including but not limited to spray bottles, nebulization, atomization or pump aerosolization of a liquid formulation, and aerosolization of a dry powder formulation, can be used.

Nasal formulations may be administered, for example, using a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation.

A useful device for intranasal administration is a small, hard bottle to which a metered dose sprayer is attached. In one embodiment, the metered dose is delivered by drawing the ketamine solution into a chamber of defined volume, which chamber has an aperture dimensioned to aerosolize and aerosol formulation by forming a spray when a liquid in the chamber is compressed. The chamber is compressed to administer the ketamine. In a specific embodiment, the chamber is a piston arrangement. Such devices are commercially available.

One preferred device for intranasal delivery of compositions and formulations is the OptiNose apparatus, which is commercially available from OptiNose US Inc. (Yardley, Pa.).

Other devices useful for administering a dose intranasally are mucosal automation device that provide atomization of topical solution across the nasal and oropharyngeal mucous membranes that produce a typical particle size of 30 microns. An example of such a device is the LMA MAD Nasal™ device (LMA Company, San Diego, Calif.), which produces a typical particle size of 30 microns, has a system dead space of 0.09 mL, a tip diameter of about 3/16″ (4 mm), and an applicator length of about 1¾″ (44 mm) can be used.

In another embodiment, intranasal drug delivery is achieved by taking a solubilized medication (liquid form) and dripping it into the nose a few drops at a time, allowing it to run down onto the nasal mucosa. This can be done using, e.g., a syringe.

In certain embodiments, the present disclosure provides liquid or powder aerosol formulations and dosage forms for intranasal administration (e.g., for use in treating subjects suffering from BPD, MDD, TRD or PTSD). In general such dosage forms contain ketamine in a pharmaceutically acceptable diluent. Pharmaceutically acceptable diluents in such liquid aerosol formulations include but are not limited to sterile water, saline, buffered saline, dextrose solution, and the like. In a specific embodiment, a diluent that may be used in the present disclosure and/or in a pharmaceutical formulation of the present disclosure is phosphate buffered saline or a buffered saline solution generally between the pH 7.0-8.0 range, or water. The present disclosure contemplates the use of any suitable pharmaceutically acceptable diluent known in the art for intranasal administration.

Formulations may also include other agents, ingredients, and/or components, e.g., that are useful for pH maintenance, solution stabilization, or for the regulation of osmotic pressure, including, but not limited to salts, such as sodium chloride, or potassium chloride, and carbohydrates, such as glucose, galactose or mannose, and the like.

Formulations for intranasal administration may include a “mucosal penetration enhancer,” i.e., a reagent that increases the rate or facility of transmucosal penetration of ketamine, such as but not limited to, a bile salt, fatty acid, surfactant or alcohol. Examples of penetration enhancers include sodium cholate, sodium dodecyl sulphate, sodium deoxycholate, taurodeoxycholate, sodium glycocholate, dimethylsulfoxide or ethanol.

Formulations disclosed herein, e.g., intranasal formulations, may include a dispersant. Preferably, a dispersant is pharmaceutically acceptable. Suitable dispersing agents are well known in the art, and include but are not limited to surfactants and the like. Such surfactants are generally used reduce surface induce aggregation caused by atomization of the solution forming a liquid aerosol. Examples of such surfactants include, but are not limited to, polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation.

In a specific embodiment, a diluent that may be used in the present invention or the pharmaceutical formulation of the present invention is phosphate buffered saline or a buffered saline solution generally between the pH 7.0-8.0 range, or water.

The liquid aerosol formulation also may optionally include pharmaceutically acceptable carriers, diluents, solubilizing or emulsifying agents, surfactants and excipients.

The aerosol formulation can be prepared as a dry powder formulation comprising a finely divided powder form of ketamine and a dispersant. For example, the dry powder formulation can comprise a finely divided dry powder containing ketamine, a dispersing agent and also a bulking agent. Bulking agents useful in conjunction with the present formulation include such agents as lactose, sorbitol, sucrose, or mannitol, in amounts that facilitate the dispersal of the powder from the device.

In another alternative embodiment, the administration comprises transdermal administration. Such treatment may be administered alone or may be supplemented with other antidepressant therapies as described herein. Transdermal administration includes passive or active transdermal or transcutaneous modalities, including, for example, patches and iontophoresis devices, as well as topical application of pastes, salves, or ointments.

Ketamine is formulated into pharmaceutical compositions comprising a carrier suitable for the desired delivery method. Exemplary carriers include, but are not limited to, any of a number of standard pharmaceutical carriers such as sterile phosphate buffered saline solutions, bacteriostatic water, and the like. A variety of aqueous carriers may be used, e.g. water, buffered water, 0.4% saline, 0.3% glycine and the like.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the methods disclosed herein.

The invention also provides a device for patient self-administration of ketamine, which device comprises a transdermal patch containing a ketamine formulation and a pharmaceutically acceptable carrier, wherein the device is formulated to disperse an amount of the ketamine formulation that contains a dose of ketamine effective to alleviate depression.

Also contemplated herein is a kit comprising a carrier for delivering a ketamine intranasally containing in close confinement therein one or more components, wherein a first component contains ketamine, a second component comprises lithium tablets or capsules and a third component comprises instructions for use.

Treatment of BPD, MDD, TRD and/or PTSD may be achieved through a method that comprises intravenous or transdermal administration of multiple doses of ketamine over a period of time ranging from a day or two up to −21 days. In specific embodiments, the ketamine is administered at least one, two, at least three, at least four, at least five, at least six, at least seven, at least eight, at least nine times in fourteen days. In other embodiments, the ketamine is administered at least one, two, at least three, at least four, at least five, at least six, at least seven, at least eight, at least nine times in twenty-one days.

Any chronic MDD or TRD may be treated by the methods described herein. Such depression may include but is not limited to any of: major depressive disorder, single episode, recurrent major depressive disorder-unipolar depression, seasonal affective disorder-winter depression, bipolar mood disorder-bipolar depression, mood disorder due to a general medical condition-with major depressive-like episode, or mood disorder due to a general medical condition-with depressive features, wherein those disorders are resistant to treatment in a given patient. Thus, any patient that presents with one of those disorders and who has not responded to an adequate trial of two antidepressant treatments in the current episode can be treated with ketamine and lithium. PTSD may also be treated using the methods described herein.

Patients with BPD, TRD, MDD or PTSD are treated with a course of ketamine (for example administered 0.5 mg/kg IV three times weekly for two weeks). Patients who respond to this treatment, are then started on a regimen of lithium carbonate dosed daily at a range of 300-1800 mg daily for the purpose of extending and maintaining the antidepressant effect of ketamine. The therapeutic effect of lithium following a response to ketamine is expected to last at least 3 months but may last up to one year or longer, as long as the lithium medication is continued.

### EXAMPLE

A single-site early phase study will enroll patients with treatment-resistant major depression (TRD) in a randomized, placebo (PLC)-controlled, double blind clinical trial of lithium (Li)+ketamine (KET). The study will efficiently test the efficacy of Li compared to PLC in continuing the antidepressant response to KET. The study occurs in several phases. During a Screening phase participants undergo informed consent followed by a thorough medical and psychiatric evaluation. Eligible participants who meet all study inclusion/exclusion criteria receive a single Test KET(ketamine) Infusion to determine antidepressant response status. Non-responders [defined as <50% improvement in depression severity as measured by the Montgomery-Asberg Depression Rating Scale (MADRS) 24 hours following the infusion] are exited from the study and provided with standard of care. KET responders (2:50% improvement in MADRS) proceed to the Li/PLC treatment phase in which participants receive up to 1200 mg/day of Li or matching PLC under double blind conditions. Participants proceed to a 4-week Continuation Phase while remaining on their treatment assignment (Li/PLC) during which time depressive symptoms are monitored twice weekly. The primary outcome is depression severity as measured by the MADRS in the Li compared to PLC group at the 2-week point during the Continuation Phase. Change in MADRS score over the remaining 2 weeks of the Continuation Phase and overall time-to-relapse during the Continuation Phase are secondary endpoints.

Patients must have a primary diagnosis of major depressive disorder (MDD) confirmed by the Structured Clinical Interview for DSM-IV Axis I Disorders (SCID) 40, and at least moderate depression severity [operationalized as a score 2: 14 on Quick Inventory of Depressive Symptomatology-Self Report (QIDS-SR) 41 and 2: 4 on the Clinical Global Impression-Severity (CGI-S) scale 42]. Participants must meet criteria for TRD, defined as an inadequate response to 2: 2 antidepressant medications determined by the Antidepressant Treatment History Form (ATHF) criteria 43. Medical Evaluation: Patients undergo a medical history, physical examination, ECG, laboratory tests (CBC, comprehensive metabolic panel, TSH), urine toxicology, and urine pregnancy test. Uncontrolled hypertension is excluded. If initial eligibility criteria are met, a taper is conducted (if necessary) such that prohibited medications are discontinued over 7 days (or longer if necessary). Concomitant Medication: Patients are required to be antidepressant drug-free for a minimum of one week (4 weeks for fluoxetine) prior to the initial test KET infusion. Contact is made with the patient's physician to confirm medication dose (s), and with the individual identified by the patient. In no case is a medication tapered if the medication is judged to be helpful for the patient based on a consensus between the patient the patient's treating provider and the study PI.

Eligible participants will undergo a single test KET infusion (0.5 mg/kg infused over 40 min). This procedure is designed to establish KET response prior to randomizing participants to Li/PLC; this design feature is to maximize study efficiency while minimizing participant burden. Based on prior studies a response rate of approximately 70% is expected. Participants arrive at the treatment center on the morning of the infusion day. A baseline depression rating is done using the MADRS prior to any intervention; this rating establishes the baseline for the entire study. An indwelling catheter is placed in the antecubital vein of the non-dominant arm, and pulse, blood pressure, digital pulse-oximetry, and ECG monitoring are instituted. An anesthesiologist is present throughout the administration of study drug, and a medical cart is available for emergencies. After the acute post-infusion period and attainment of sufficient recovery, the patient is given a meal and allowed to rest. Safety assessments are conducted throughout the day in order to assess for psychotomimetic [Brief Psychiatric Rating Scale (BPRS+) 45], dissociative [Clinician-Administered Dissociative States Scale (CADSS) 46] and general side effects [Patient Rated Inventory of Side Effects (PRISE) 47]. At 240 minutes post-infusion, a physical examination and check of vital signs is performed. If stable, the patient is discharged home in care of a family member or friend to escort them home. Participants return to the outpatient clinic the following day (Visit 0, Day 0) for safety and efficacy assessments. Participants who do not demonstrate 2:50% reduction in depression severity (i.e., change in MADRS score from baseline) are exited from the study and provided with standard of care.

24 hours following the test KET infusion, participants are classified as responders or non-responders as described above. Only responders are randomized that morning to a schedule of Li or matching PLC and titrated as follows: Li 300 mg/PLC qhs×1 day, then Li 600 mg/PLC qhs×1 day, then Li 900 mg/PLC qhs for the remainder of the study. Additional adjustments of Li doses (up to 1200 mg/day) will be allowed to achieve therapeutic Li blood levels (2:0.5 mEq/L) for participants randomized to Li.

Participants are monitored twice weekly as outpatients throughout the 4-week Continuation Phase. The therapeutic effect of lithium following ketamine may last up to one year or longer, as long as the lithium medication is continued. It is anticipated that Responders to ketamine that receive lithium will maintain the ketamine anti-depressant effect for at least two or three months after receiving the last dose of ketamine.

1. The method of treating a patient afflicted with treatment resistant depression which comprises administering to the patient an effective amount for treating the treatment resistant depression of ketamine, determining that the patient has responded to the ketamine, and thereafter administering lithium to the patient.

2. The method of claim 1 wherein the effective amount of ketamine comprises between about 0.1 m/kg to about 1.0 mg/kg per day.

3. The method of claim 1 which comprises intranasally administering the ketamine.

4. The method of claim 1 which comprises intravenously administering the ketamine to the patient.

5. The method of claim 1 which comprises administering between about 300 mg/day and about 1800 mg/day of lithium to the patient.

6. The method of claim 1 wherein response to administration of ketamine in measured with the Montgomery-Asberg Depression Rating Scale (MADRS).

7. The method of claim 6 wherein response to the ketamine comprises at least a fifty percent improvement in depression severity within twenty four hours after administration of ketamine using the MADRS scale.

8. The method of claim 1 which comprises conducting the determining step at least twenty four hours after administration of the ketamine.

9. The method of claim 1 wherein the lithium comprises lithium carbonate.

10. The method of claim 1 wherein the lithium is administered in a divided daily dose.

11. The method of claim 1 which comprises continuing daily administration of lithium to the patient until there is at least a fifty percent increase in the patient's MADRS score and thereafter administering at least one dose of an effective amount of ketamine for treating the treatment resistant depression to the patient.

12. The method of treating treatment resistant depression which comprises maintaining the improvement in severity of treatment resistant depression in a patient that displays such improvement after administration of ketamine to the patient by administering a daily dose of lithium to the patient.

13. The method of treating post-traumatic stress disorder which comprises administering to the patient an effective amount of ketamine for treating the post-traumatic stress disorder, determining that the patient has responded to the ketamine, and thereafter administering lithium to the patient.

14. The method of claim 13 wherein the effective amount of ketamine comprises between about 0.1 m/kg to about 1.0 mg/kg per day.

15. The method of claim 13 which comprises continuing daily administration of lithium to the patient until there is at least a fifty percent increase in the patient's CAPS score and thereafter administering at least one dose of an effective amount of ketamine for treating the post-traumatic stress disorder to the patient.

16. The method of treating a patient afflicted with major depressive disorder which comprises administering to the patient an effective amount of ketamine for treating the major depressive disorder, determining that the patient has responded to the ketamine, and thereafter administering lithium to the patient.

17. The method of claim 16 wherein the effective amount of ketamine comprises between about 0.1 m/kg to about 1.0 mg/kg per day.

18. The method of claim 16 which comprises continuing daily administration of lithium to the patient until there is at least a fifty percent increase in the patient's MADRS score and thereafter administering at least one dose of an effective amount of ketamine for treating the major depressive disorder to the patient.

19. The method of claim 16 wherein the lithium comprises lithium carbonate.

20. The method of claim 14 wherein the lithium comprises lithium carbonate.

21. The method of claim 13 wherein response to administration of ketamine in measured with the Clinician Administered PTSD Scale (CAPS).

22. The method of treating a patient afflicted with bipolar depression which comprises administering to the patient an effective amount of ketamine for treating the bipolar depression, determining that the patient has responded to the ketamine, and thereafter administering lithium to the patient.

23. The method of claim 22 wherein the effective amount of ketamine comprises between about 0.1 m/kg to about 1.0 mg/kg per day.

24. The method of claim 23 wherein the lithium comprises lithium carbonate.

25. The method of claim 22 which comprises continuing daily administration of lithium to the patient until there is at least a fifty percent increase in the patient's MADRS score and thereafter administering at least one dose of an effective amount of ketamine for treating the major depressive disorder to the patient.

26. The method of claim 1 which comprises administering between about 600 mg/day and about 1200 mg/day of lithium to the patient.
